SEQUENCE LISTING (4 sequences)

SEQ ID NO: 1 - RNA, 429 nt, synthetic construct
  source 1..429: mol_type = other RNA
gggcgataatacagttttggactcaggtgtgagattttatgatcaggactatgaaggacaaataaccccaatggaatatgtaactgggttgtataacttttggtcagggccaatagagttacgttttgattttgtttcaaatgcgtttcacactggaacagtgattatatcagcggagtataatcgatcatctactaatacggatgagtgtcagtcacactcaacttatactaaaacgttccacttgggagaacaaaaatcagtacatttcactgtgccttatatatatgatactgttatgcggagaaatacggctagcgcctatttaccggtaactgattatgataaggcagataatgttagtagggcgcaggctacggggattagagcagaatctaaaatgagagtgaaagtgagatcgccctatag

SEQ ID NO: 2 - RNA, 429 nt, synthetic construct
  source 1..429: mol_type = other RNA
gggcgatctcactttcactctcattttagattctgctctaatccccgtagcctgcgccctactaacattatctgccttatcataatcagttaccggtaaataggcgctagccgtatttctccgcataacagtatcatatatataaggcacagtgaaatgtactgatttttgttctcccaagtggaacgttttagtataagttgagtgtgactgacactcatccgtattagtagatgatcgattatactccgctgatataatcactgttccagtgtgaaacgcatttgaaacaaaatcaaaacgtaactctattggccctgaccaaaagttatacaacccagttacatattccattggggttatttgtccttcatagtcctgatcataaaatctcacacctgagtccaaaactgtattatcgccctatag

SEQ ID NO: 3 - DNA, 18 bp, synthetic construct
  source 1..18: mol_type = other DNA
taatacgactcactatag

SEQ ID NO: 4 - DNA, 533 bp, synthetic construct
  source 1..533: mol_type = other DNA
cggtacctcgcgaatgcatctagatatcggatcctaatacgactcactatagggcgataatacagttttggactcaggtgtgagattttatgatcaggactatgaaggacaaataaccccaatggaatatgtaactgggttgtataacttttggtcagggccaatagagttacgttttgattttgtttcaaatgcgtttcacactggaacagtgattatatcagcggagtataatcgatcatctactaatacggatgagtgtcagtcacactcaacttatactaaaacgttccacttgggagaacaaaaatcagtacatttcactgtgccttatatatatgatactgttatgcggagaaatacggctagcgcctatttaccggtaactgattatgataaggcagataatgttagtagggcgcaggctacggggattagagcagaatctaaaatgagagtgaaagtgagatcgccctatagtgagtcgtattagtcgactgcagaggcctgcatgcaagcttggcgtaatcatg

(19) United States
(12) Patent Application Publication
(10) Pub. No.: US 2026/0117272 A1
(43) Pub. Date: Apr. 30, 2026

(54) LARGE-SCALE PRODUCTION METHOD OF DOUBLE-STRANDED RNA

(71) Applicant: Na Vaccine Institute, Seoul (KR)

(72) Inventors: Dong Ho KIM, Seoul (KR); Seung Bin CHA, Seoul (KR); Seung Beom HONG, Seoul (KR); Seung Kwan CHOI, Seoul (KR)

(73) Assignee: Na Vaccine Institute, Seoul (KR)

(21) Appl. No.: 19/362,665

(22) Filed: Oct. 20, 2025

(30) Foreign Application Priority Data
Oct. 25, 2024 (KR) 10-2024-0147625

(51) Int. Cl. C12P 19/34

(52) U.S. Cl. CPC C12P 19/34

(57) ABSTRACT

Provided is a method for large-scale production of double-stranded RNA using PCR.

### CROSS-REFERENCE TO RELATED APPLICATION

This application is based on and claims priority under 35 USC § 119 to Korean Patent Application No. 10-2024-0147625, filed on Oct. 25, 2024, in the Korean Intellectual Property Office, the disclosure of which is incorporated by reference herein in its entirety.

### REFERENCE TO A SEQUENCE LISTING

A Sequence Listing, submitted as an XML file and compliant with WIPO Standard ST.26, forms part of the present application. The Sequence Listing is identified as follows: File name “559886US.ST26.xml,” created on Oct. 16, 2025, 2025, with a size of 5,738 bytes.

### BACKGROUND

### 1. Field

The disclosure relates to a method for large-scale production of double-stranded RNA.

### 2. Description of the Related Art

Double-stranded RNA (dsRNA) is utilized in various fields, such as pharmaceuticals and research reagents, as a tool capable of suppressing the expression of a specific gene through an RNA interference (RNAi) mechanism, or as a TLR3 agonist.

The production of RNA has mainly been based on recombinant DNA technology using Escherichia coli culture. In such production methods, a plasmid containing the desired sequence is introduced into bacteria, the bacteria are then mass-cultured, the plasmid DNA is extracted, and RNA is produced by performing in vitro transcription (IVT) using the plasmid DNA as a template. This conventional technology has primarily been used to produce long mRNA exceeding 1,000 bp in length, and it does not employ PCR.

However, this conventional technology requires considerable cost and time in the process of culturing large amounts of E. coli. Specifically, a large amount of culture medium, which is essential for cell growth and maintenance, is required during mass culture of bacteria into which the recombinant vector has been introduced, resulting in high cost during mass culture. In addition, the culture time requires at least five days, and after a single large-scale production, any subsequent production must be restarted from the beginning of bacterial culture with the recombinant vector, leading to poor time efficiency.

Meanwhile, siRNA has generally been produced by direct synthesis. However, direct synthesis is applicable only when the length of the dsRNA is short, and it is unsuitable for producing dsRNA exceeding 300 bp in length.

Double-stranded RNA may be used as a pharmaceutical or research reagent, for example as a TLR3 agonist, and thus there is demand for its large-scale production. However, as described above, apart from the bacterial mass-culture method that does not use PCR, there has been no method for large-scale production of dsRNA.

Conventional PCR methods typically use a small reaction volume (about 10 μL to about 200 μL). To perform high-volume PCR, it has generally been necessary to employ thermal cycler systems that process multiple small reaction volumes. Such thermal cycler system PCRs are optimized for processing small volumes, and are therefore unsuitable for industrial-scale, high-volume PCR. Furthermore, because they are designed for specific volumes or formats, additional equipment is required to accommodate new applications or large-scale reactions. These systems also have disadvantages in that they are expensive due to the precision equipment required, and they require regular and complex maintenance.

Accordingly, there is a need to develop a method for large-scale production of double-stranded RNA that incorporates a new large-scale PCR system, so that large amounts of double-stranded RNA may be produced more cost-effectively, efficiently, and rapidly than with the conventional technologies.

### SUMMARY

Provided is a method for large-scale production of double-stranded RNA, including (a) preparing a recombinant vector including a DNA fragment having a sequence including an RNA polymerase promoter sequence linked to each 5′ end of a DNA sequence encoding a target nucleotide sequence, (b) performing PCR using the recombinant vector as a template to obtain an amplified DNA fragment product, (c) performing an in vitro transcription reaction using the DNA fragment product as a template to obtain a first double-stranded RNA product including an RNA polymerase promoter sequences at both ends, and (d) treating the obtained first double-stranded RNA product including the RNA polymerase promoter sequence with a DNA-degrading enzyme and a single-strand-specific RNase to obtain a second double-stranded RNA.

Provided is a double-stranded RNA prepared by the method.

Additional aspects will be set forth in part in the description which follows and, in part, will be apparent from the description, or may be learned by practice of the presented embodiments of the disclosure.

According to an aspect of the disclosure, a method for large-scale production of double-stranded RNA, includes (a) preparing a recombinant vector comprising a DNA fragment having a sequence including an RNA polymerase promoter sequence linked to each 5′ end of a DNA sequence encoding a target nucleotide sequence, (b) performing PCR using the recombinant vector as a template to obtain an amplified DNA fragment product, (c) performing an in vitro transcription reaction using the DNA fragment product as a template to obtain a first double-stranded RNA product including an RNA polymerase promoter sequences at both ends, and (d) treating the obtained first double-stranded RNA product including the RNA polymerase promoter sequence with a DNA-degrading enzyme and a single-strand-specific RNase to obtain a second double-stranded RNA.

The disclosure provides a method that overcomes the shortcomings of the conventional method of producing double-stranded RNA through large-scale cultivation of E. coli and extraction of plasmid therefrom. According to the disclosure, by simply establishing a cell bank, a large amount of double-stranded RNA may be rapidly manufactured using only PCR in a subsequent step, resulting in a very large cost savings compared to conventional technologies.

The method includes (a) preparing a recombinant vector including a DNA fragment having a sequence including an RNA polymerase promoter sequence linked to each 5′ end of a DNA sequence encoding a desired nucleotide sequence.

The desired nucleotide sequence includes a double-stranded RNA. The double-stranded RNA may have 3′ overhangs at both ends. The double-stranded RNA may have a length of 300 nt or more, 500 nt or more, 800 nt or more, 1000 nt or more, 1300 nt or more, or 1500 nt. For example, the double-stranded RNA may have a length of about 300 nt to about 1000 nt, about 500 nt to about 1000 nt, about 800 nt to about 1000 nt, about 1000 nt to about 1300 nt, or about 1000 nt to about 1500 nt.

The double-stranded RNA may be a TLR3 agonist. The double-stranded RNA may act as a TLR3 ligand.

As used herein, the term “TLR agonist” refers to an agonist that binds to and activates a toll-like receptor (TLR) expressed in a mammalian cell (e.g., a human cell). A TLR3 agonist binds to and activate TLR3.

As used herein, the first double-stranded RNA refers to an RNA having a structure in which a complementary dsRNA (double-stranded RNA) portion having an arbitrary length and an arbitrary sequence is located, and a long single-stranded RNA (single-stranded RNA, ssRNA) is bound to both 3′ ends of the dsRNA. The first double-stranded RNA is also referred to as a heterostructured RNA (hsRNA).

As used herein, the second double-stranded RNA refers to an RNA having a structure in which a complementary dsRNA (double-stranded RNA) portion having an arbitrary length and an arbitrary sequence is located, and a single-stranded RNA is bound to both 3′ ends of the dsRNA in a length of 10 bp or less.

The double-stranded RNA may include a first strand including 5′-end, a 3′-end and a region complementary to the second strand; a second strand including a 5′-end, a 3′-end and a region complementary to the first strand; a first overhang positioned at the 3′-end of the first strand having one or more consecutive ribonucleotides; and a second overhang positioned at the 3′-end the second strand having one or more consecutive ribonucleotides. In the first strand and the second strand, the complementary region may independently be derived from a non-human subject. The first overhang and the second overhang may have about 1 nt to about 10 nt.

The first and second overhangs may each have the sequence UAUAG. That is, the complementary region may independently include the sequence UAUAG at the 3′ end.

TLR3 is normally expressed in endosomal compartments of dendritic cells (DCs), B cells, monocyte-derived macrophages, and many tumor tissues, and detects dsRNA. The dsRNA may be derived from viruses, bacteria, or abnormal cells.

When the TLR3 receptor recognizes dsRNA, it stimulates the secretion of type I interferon and proinflammatory cytokines. DCs are activated into mature antigen-presenting cells (APCs), where antigen epitopes are loaded onto MHC-1 molecules and presented to naive T cells. Activation of DCs by the TLR3 enzyme not only contributes to the induction of innate and adaptive immune responses against microbial pathogens, but also activates CD8+ T cells and natural killer (NK) cells. The minimum length of dsRNA that may bind to TLR3 and induce dimerization may be 45 mer.

The double-stranded RNA, when administered to a subject, may promote an immune response in the subject.

In the first strand and the second strand, the region complementary to the second strand may be complementary to the region complementary to the first strand. In the first strand and the second strand, the complementary region may have nucleotide sequence complementarity of 75% or more, 80% or more, 85% or more, 90% or more, 95% or more, or 100% between the nucleotide sequences of the first strand and the second strand.

The complementary region may have complementary nucleotides at positions where all nucleotides correspond between the nucleotide sequences of the first strand and the second strand.

In the first strand and the second strand, the complementary region may independently not include a sequence complementary to a nucleotide sequence of the human genome.

In the first strand and the second strand, the complementary regions may be independently derived from a non-human subject. The subject may be of viral, bacterial, or plant origin. The virus may be Sacbrood virus (SBC). The double-stranded RNA molecule may be derived from the VP1 protein gene of Sacbrood virus.

In the first strand and the second strand, the first overhang and the second overhang may not contain complementary sequences to each other. The first overhang and the second overhang may not contain complementary sequences, such that do not form a double strand.

In the first strand and the second strand, the first overhang and the second overhang may be independently obtained by cleavage of an RNA molecule including a double-stranded region and a single-stranded region using an RNase, for example, RNase T1. In the first strand and the second strand, the first overhang and the second overhang may be independently obtained by hybridizing a pre-first strand having nucleotides extended at the 3′-end relative to the first strand and a pre-second strand having nucleotides extended at the 3′-end relative to the second strand, and then contacting the hybridized product with an RNase, for example, RNase T1, to digest the ssRNA at the 3′-end.

The RNase may be an endoribonuclease or exoribonuclease that specifically cleaves single-stranded RNA. The RNase T1 may be an endoribonuclease that specifically cleaves single-stranded RNA at the G residue. The RNase T1 may cleave the phosphodiester bond between the 3′-guanyl residue and the 5′-OH residue of the neighboring nucleotide. The reaction product may be a polynucleotide having 3′-GMP and a terminal 3′-GMP.

The first strand and the second strand may have the nucleotide sequences of SEQ ID NO: 1 and SEQ ID NO: 2, respectively.

In the first strand and the second strand, the 5′-end may independently not have a 5′-cap.

In the first strand and the second strand, the 5′-end may independently have a triphosphate, a diphosphate, or a monophosphate.

In the first strand and the second strand, the 3′-end may independently have a phosphate.

In the first strand and the second strand, the 3′-end may independently have a hydroxyl group.

The RNA polymerase may be of bacterial or viral origin. The RNA polymerase may be T7 RNA polymerase. The promoter may be a T7 promoter. The T7 promoter may be full length or a portion thereof, for example, the core sequence.

The DNA fragment may be double-stranded.

The vector can be any known vector. The vector may be of bacterial, viral, or phage origin. The vector may be a plasmid or a plasmid-derived vector.

The method includes (b) performing PCR using the recombinant vector as a template to obtain the amplified DNA fragment product.

The PCR is used in its generally known sense. The PCR may include, for example, repeating denaturation, annealing, and extension. The PCR reaction may be a thermal cycling reaction in which reactions at each temperature suitable for denaturation, annealing, and extension reactions are repeated.

In (b), PCR may be performed by a large-scale PCR system.

The large-scale PCR system may include a reaction vessel made of a heat transferable material containing a PCR reaction mixture, and a first heat transfer vessel, a second heat transfer vessel, and a third heat transfer vessel, each capable of accommodating the reaction vessel and containing a liquid set to a temperature for denaturation, annealing, and extension, respectively.

The temperature for denaturation, annealing, and extension may be a temperature required to dissociate the DNA fragment into single strands, a temperature required to anneal the dissociated single strands with a primer, and a temperature suitable for extension of the DNA strand by a DNA polymerase. Such temperatures may be appropriately selected depending on the characteristics of the selected DNA, primer, and DNA polymerase. The DNA polymerase may be a thermostable DNA polymerase. The DNA polymerase may be Taq DNA polymerase.

The PCR may include moving the reaction vessel to a first heat transfer vessel, a second heat transfer vessel, and a third heat transfer vessel to perform denaturation, annealing, and extension reactions, respectively.

The reaction vessel may be made of a material capable of transferring heat of the liquid in the heat transfer vessel to the PCR reaction mixture in the reaction vessel when in contact with the liquid in the heat transfer vessel. The large-scale PCR system may include one or more reaction vessels. The large-scale PCR system may include, for example, about 1 to about 10, about 1 to about 8, or about 1 to about 6 reaction vessels.

The heat transfer vessel may be capable of accommodating two or more of the reaction vessels. The heat transfer vessel may accommodate, for example, about 1 to about 10, about 1 to about 8, or about 1 to about 6 reaction vessels. The heat transfer vessel may accommodate the reaction vessel within the liquid contained therein. Accordingly, thermal cycling reactions may be performed simultaneously in a plurality of reaction vessels.

The reaction vessel may be capable of accommodating a large volume of PCR reaction mixture. The volume may be 1 ml or more, 10 ml or more, or 100 ml or more. The volume may be about 100 ml to about 1000 L, about 100 ml to about 100 L, about 100 ml to about 10 L, about 100 ml to about 900 ml, about 100 ml to about 700 ml, about 100 ml to about 500 ml, about 100 ml to about 300 ml, about 100 ml to about 200 ml, about 300 ml to about 900 ml, about 300 ml to about 700 ml, about 200 ml to about 500 ml, or about 300 ml to about 500 ml. In an embodiment, the reaction vessel may contain 100 ml or more of PCR reaction mixture.

The reaction vessel may be made of plastic, for example, polypropylene, polyethylene, or metal. The reaction vessel may be flexible, such as vinyl. In an embodiment, the reaction vessel may be a plastic sealed bag. The bag may be flexible, have a predetermined length, and may be sealed by compressing a portion, for example, by tying with a fixing means.

The liquid in the heat transfer vessel may be an aqueous liquid. The aqueous liquid may be a solution containing water. The liquid may be water. The heat transfer vessel may be in the form of a tank, a bath, or a chamber.

The heat transfer vessel may include a heating means for maintaining the liquid contained therein at a set temperature. The heating means may be an electric heating means. The heating means may be a heating block.

The heat transfer vessel may further include a controller that regulates heat transfer from the heating means so as to maintain the liquid contained therein at a set temperature.

Additionally, the large-scale PCR system may further include a transport means for transporting the reaction vessel so that it is immersed in a liquid in each of the heat transfer vessels. The transfer means may be configured to maintain the reaction vessel in the heat transfer vessel for a time required for the denaturation, annealing and extension reactions, respectively, and then transfer the reaction vessel to the heat transfer vessel of the next process. The large-scale PCR system may include a control means for maintaining the reaction vessel in each heat transfer vessel for a time required for denaturation, annealing and extension reactions, and then transferring the reaction vessel to the heat transfer vessel of the next process.

In (b), the PCR may include:
initially denaturing the PCR reaction product at about 95° C. to about 96° C. for 5 minutes;
repeating denaturation, annealing and extension reactions 35 times to 38 times under the conditions of about 95° C. to about 96° C. for about 1 minute to about 1 minute 30 seconds, about 58° C. to about 62° C. for about 1 minute 10 seconds to about 1 minute 40 seconds, and about 70° C. to about 74° C. for about 1 minute to about 1 minute 20 seconds for the initially denatured product; and performing a final treatment at about 71° C. to about 76° C. for 5 minutes.

The PCR may use the RNA polymerase promoter or a sequence complementary thereto as a primer.

In the method, after (a) and before (b), a process of introducing the recombinant vector into a microbial cell and culturing the same to construct a cell bank (CB) containing the recombinant vector may be further included.

The microbial cells may be a bacterial cell or a fungal cell. The bacterial cell may be Escherichia coli. The fungal cell may be a yeast cell.

The method includes (c) performing an in vitro transcription reaction using the DNA fragment product as a template to obtain a double-stranded RNA product including an RNA polymerase promoter sequence at both ends.

The in vitro transcription reaction (IVT) may be a transcription reaction performed in vitro. The in vitro transcription reaction may be performed using the amplified DNA product as a template and in the presence of RNA polymerase. The in vitro transcription reaction may be performed, for example, in a reaction solution containing linear template DNA, NTPs, and an RNA polymerase, such as T7 RNA polymerase. The reaction may be carried out in the presence of a suitable buffer or salt. The buffer used in the in vitro transcription reaction may include 5× Reaction buffer (0.5 M HEPES, magnesium chloride hexahydrate and distilled water) and a Dialysis buffer (sodium chloride, 1 M Tris-HCl, glycerin and distilled water).

The buffer used in the in vitro transcription reaction may have a pH of about 7.5 to about 8.0. If the pH of the buffer is less than 7.5 or greater than 8.0, the efficiency of the IVT synthesis reaction is reduced, thereby lowering the yield of double-stranded RNA.

The method includes (d) treating the obtained first double-stranded RNA product including RNA polymerase promoter sequence with a DNA-degrading enzyme and a single-strand-specific RNase to obtain a second double-stranded RNA.

The treatment may include incubating the obtained first double-stranded RNA product in the presence of a DNA-degrading enzyme to degrade DNA present in the first double-stranded RNA product.

Additionally, the treatment may further include incubating the double-stranded RNA product treated with a DNA-degrading enzyme in the presence of a single-strand-specific RNase to produce a second double-stranded RNA having 3′ overhangs at both ends.

The single-strand-specific RNase may be an endoribonuclease or exoribonuclease that specifically cleaves single-stranded RNA. The single-strand-specific RNase may be RNase T1. The RNase T1 may be an endoribonuclease that specifically cleaves single-stranded RNA at G residues. The RNase T1 may cleave the phosphodiester bond between the 3′-guanyl residue and the 5′-OH residue of the neighboring nucleotide. The reaction product may be a polynucleotide having 3′-GMP and a terminal 3′-GMP.

The method may further include purifying the second double-stranded RNA obtained in (d). The purification may be performed by a known method. The purification may include one or more of precipitation, centrifugation, filtration, ion exchange chromatography, and electrophoresis.

The concentration of the second double-stranded RNA obtained by the method may be 6 mg/ml or more.

According to another aspect of the disclosure, a double-stranded RNA is prepared by the method.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described in more detail through examples. However, these examples are intended to exemplify the present disclosure and the scope of the present disclosure is not limited to these examples.

### Example: Large-Scale Production of Double-Stranded RNA Including PCR and In Vitro Transcription

### (1) Synthesis of DNA Fragment

A DNA fragment was synthesized in which a sequence including a T7 promoter sequence (SEQ ID NO: 3) was linked to each 5′ end of the DNA sequence encoding the nucleotide sequences of SEQ ID NO: 1 and SEQ ID NO: 2. The DNA fragment is a dsDNA formed by the nucleotide sequence of SEQ ID NO: 4 and its complementary sequence.

### (2) Bacterial Culture and Establishment of Cell Bank

The DNA fragment was ligated into the PUC19 (Thermo Fisher, Cat SD0061) vector cut with Smal to produce a recombinant vector. The recombinant vector was then introduced into E. coli DH5a by a transformation. The resulting recombinant E. coli was cultured in LB/amp medium to proliferate E. coli containing the recombinant vector, thereby producing a research cell bank (RCB). One vial of RCB was taken out and the recombinant E. coli sample was thawed in a clean bench.

The RCB was confirmed to have been thawed. 1 mL of RCB was inoculated into a 0.5 L flask containing 100 mL of medium. 96 μL of kanamycin (52 mg/mL) stock was added thereto. The cell-containing medium was cultured in a shaking incubator at 37° C. and 200 rpm. The growth curve of E. coli cells was monitored during seed culture. The seed culture broth with an O.D₆₀₀ value of 1.2 was stored in a clean bench.

The seed culture broth with O.D_{600=1.2} stored in the clean bench was confirmed. 5 mL of the seed culture was inoculated into each 2 L flask containing 500 mL of medium. 481 μL of kanamycin (52 mg/mL) stock was added thereto. The cell-containing medium was cultured in a shaking incubator at 37° C. and 200 rpm. The growth curve of E. coli cells was confirmed during the main culture.

Subsequently, the RCB was used to prepare a master cell bank (MCB).

### (3) Recovery of PCR Reaction Mixture

The E. coli cells proliferated in the MCB culture broth were separated from the supernatant, and the recombinant vector was isolated from the E. coli by the alkaline lysis method (Qiagen Midi Prep Kit).

The completed culture broth was divided into 200 mL portions and centrifuged at 4° C., 4000 rpm for 15 minutes. 10 mL of P1 buffer was added to resuspend the pellet. 10 mL of P2 buffer was added, and the tube was gently inverted 4 to 6 times to mix the buffer uniformly, followed by incubation for 5 minutes. 10 mL of cooled P3 buffer was added, and the tube was gently inverted 4 to 6 times to mix the buffer uniformly. The mixture was allowed to stand on ice for 20 minutes. The mixture was centrifuged at 4° C. for 30 minutes, and the supernatant was collected. The recovered supernatant was centrifuged at 4° C. for 15 minutes under conditions of 20,000 g or more. After centrifugation, the supernatant was collected.

A QIAGEN-tip 500 column was equilibrated with 10 mL of QBT buffer. The buffer was allowed to drain from the column by gravity. The collected supernatant from above was loaded onto the QIAGEN-tip. The column was washed twice with 30 mL of QC buffer. The plasmid was eluted with 15 mL of QF buffer. 10.5 mL of Isopropanol (0.7 volumes) was added at room temperature to precipitate the plasmid. The mixture was centrifuged at 4° C. for 30 minutes under conditions of 15,000 g or more. The plasmid pellet was washed using 5 mL of 70% ethanol. After centrifugation at 4° C. for 10 minutes under conditions of 15,000 g or more, the supernatant was carefully removed. The pellets were dried at room temperature for 5 minutes to 10 minutes. The pellet was dissolved using TE buffer (pH 8.0) or 10 mM Tris buffer (pH 8.5).

As a result, recombinant vector DNA serving as template DNA for in vitro transcription (IVT) based on T7 polymerase was obtained.

### (4) Obtaining DNA Product by PCR Amplification Using the Isolated Recombinant Vector as a Template

The template DNA for in vitro transcription (IVT) based on T7 polymerase was amplified by PCR using the recombinant vector DNA as a template.

PCR conditions are as follows. One cycle was performed at 95.8° C. for 5 minutes, and then a thermal cycle consisting of 95.8° C. for 80 seconds, 61° C. for 95 seconds, and 73° C. for 74 seconds was repeated 36 times. Finally, the PCR reaction was terminated by maintaining the reaction mixture at 74.5° C. for 5 minutes, and the PCR reaction mixture was obtained.

The PCR was performed using a large-scale PCR system. The large-scale PCR system includes a reaction vessel made of a heat-transferable material containing a PCR reaction mixture, and a first heat transfer vessel, a second heat transfer vessel, and a third heat transfer vessel, each capable of accommodating the reaction vessel and containing a liquid set to a temperature for denaturation, annealing, and extension, respectively. The heat transfer vessel may include a heating means for maintaining the liquid contained therein at a set temperature. The heating means may be an electric heating means. The heating means may be a heating block.

The heat transfer vessel may further include a controller that regulates heat transfer from the heating means to maintain the liquid contained therein at a set temperature.

Additionally, the large-scale PCR system includes a transfer means for transferring the reaction vessels so as to be immersed in the liquid in each of the heat transfer vessels. The transfer means maintains the reaction vessel in the heat transfer vessel for the time required for the denaturation, annealing and extension reactions, respectively, and then transfers it to the heat transfer vessel of the next process. The large-scale PCR system includes a control means for maintaining the reaction vessel in the heat transfer vessel for a time required for denaturation, annealing and extension reactions, respectively, and then transferring the reaction vessel to the heat transfer vessel of the next process.

In this embodiment, the reaction vessel is a sealing bag, and the first heat transfer vessel, the second heat transfer vessel, and the third heat transfer vessel are tank 1, tank 2, and tank 3 containing water. The PCR includes moving the reaction vessel to a first heat transfer vessel, a second heat transfer vessel, and a third heat transfer vessel to perform denaturation, annealing, and extension reactions, respectively. The reaction vessel is made of a material capable of transferring heat of the liquid in the heat transfer vessel to the PCR reactants in the reaction vessel when in contact with the liquid in the heat transfer vessel. The specific PCR process is as follows.

PCR tanks 1, 2, and 3 were prepared at temperatures of 95.8° C., 61° C., and 73° C., respectively. An autoclaved beaker (1 L) was prepared to carry out the PCR reaction. The PCR reaction mixture was stirred at room temperature at 180 rpm to 220 rpm for at least 3 minutes using a magnetic stirrer.

100 mL of PCR reaction mixture was transferred to each of six sealed bags (GMP grade), which were then sealed. Each sealed bag was fixed into a pre-set autotrailer PCR system, i.e., the large-scale PCR system, and the reaction was started. For 5 L of IVT reaction, 500 mL of PCR reaction mixture was required, and to obtain this PCR volume, two PCR reactions of 600 mL each (100 mL×6 bags) were performed.

After completion of the two PCR reactions, the 600 mL of PCR reaction mixture (100 mL×6 bags) was pooled into a 1 L bottle and stored at refrigerated conditions.

### (5) In Vitro Transcription (IVT)

Using the amplified DNA product as a template, an in vitro transcription reaction (IVT) was performed using a MEGAscript™ T7 Transcription Kit (Thermo Fisher, cat AMB13345). The detailed procedure of the in vitro transcription reaction are as follows.

A 37° C. constant temperature water bath was preheated at least 1 hour before use. The template DNA, i.e. PCR product, rNTP mixture, TC buffer (5× reaction buffer) (0.5 M HEPES, magnesium chloride hexahydrate, and distilled water), Dialysis buffer (sodium chloride, 1 M Tris-HCl, glycerin, and distilled water), and 1 M spermidine were mixed in a 5 L bottle and stirred at 750 rpm to 800 rpm. After stirring was complete, the mixture was pre-warmed by incubation in a 37° C. constant temperature water bath. DTT and enzymes were placed in the 5 L bottle.

The IVT mixture in the 5 L bottle was stirred at 750 rpm to 850 rpm at room temperature. The 5 L bottle containing the IVT mixture was incubated in the 37° C. water bath for 13 hours to 20 hours to perform the IVT reaction. An 80° C. constant temperature water bath was preheated for at least 1 hour before use. To terminate the reaction, the mixture was heat treated in an 80° C. constant temperature water bath for 20 minutes, and then cooled in a clean booth for 30 minutes. The IVT reaction mixture was mixed at 750 rpm to 800 rpm for 5 minutes (±1 minute).

By the IVT, the first strand and the second strand were simultaneously synthesized from both strands and hybridized to form the first double-stranded RNA, i.e., a heterostructured RNA.

(6) Treatment with DNase and RNase T1

The IVT product stored at room temperature was stirred at 750 rpm to 800 rpm for 5 minutes (±1 minute). After stirring, the mixture was incubated in a 37° C. water bath for 40 minutes (±5 minutes). DNase I was added to a final concentration of 0.00077 units/μL, and the mixture was stirred at 800 rpm for 5 minutes (±1 minute). The DNase I-treated IVT product bottle was reacted in a 37° C. constant temperature water bath for 13 hours to 17 hours.

After the DNase I reaction was completed, RNase T1 equivalent to 1/20,000 times the original solution was added to the bottle and stirred at 750 rpm to 800 rpm for 5 minutes (±1 minute). After stirring, the mixture was incubated in a 37° C. water bath for 1 hour to 3 hours (±5 minutes).

After the reaction was completed, the RNase T1 treatment solution was heated in an 80° C. constant temperature water bath for 20 minutes (±1 minute) and then cooled in a clean booth for 30 minutes (±1 minute). To minimize sediment in the IVT product, 5 L was divided into six 1 L centrifuge bottles (5,000 mL/6 bottles=approximately 833 mL each, 850 g, density 1.02 g/mL) and centrifuged. After centrifugation, the clear supernatant was transferred to a new 5 L bottle.

### (7) Purification and Filtration to Obtain Second Double-Stranded RNA

Using the IVT product containing only the clear supernatant, purification and filtration were performed. A nucleic acid precipitation method using isopropanol was employed to isolate double-stranded RNA having 3′ overhangs at each 3′ end from the reaction mixture.

The double-stranded RNA may have a phosphate group at the 5′ end and a hydroxyl group at the 3′ end. The phosphate may be a triphosphate, diphosphate, or monophosphate. The double-stranded RNA may not have a 5′-cap. The double-stranded RNA may have a triphosphate at the 5′ end and a hydroxyl group at the 3′ end.

As a result, approximately 20 g of double-stranded RNA was obtained from 5 L of culture. The purity of dsRNA was approximately 99.5% to 99.7%.

According to an aspect, the method for large-scale production of double-stranded RNA amplifies the desired DNA sequence using a large-scale PCR system to produce double-stranded RNA. Therefore, compared with the conventional method using large-scale bacterial culture, the consumption of medium and other consumables may be minimized, which significantly reduces manufacturing costs. In addition, the large-scale PCR system may be easily scaled up by increasing the capacity of the reaction vessel and heat transfer vessel.

Furthermore, once a cell bank is established by introducing a recombinant vector into microbial cells and culturing them, desired DNA may be continuously obtained from the cell bank and used in PCR reactions to produce double-stranded RNA, thereby providing an efficient production process.

In terms of production time, the conventional method requires more than 5 days for culture, whereas the present disclosure enables production within 24 hours by using PCR, which is a significant advantage.

In addition, by optimizing the efficiency of the IVT synthesis reaction through pH control of the buffer in the in vitro transcription (IVT) process, higher yields of double-stranded RNA may be obtained compared to conventional technologies, thereby providing higher productivity.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

1. A method for large-scale production of double-stranded RNA, comprising:
(a) preparing a recombinant vector comprising a DNA fragment having a sequence including an RNA polymerase promoter sequence linked to each 5′ end of a DNA sequence encoding a desired nucleotide sequence;
(b) performing PCR using the recombinant vector as a template to obtain an amplified DNA fragment product;
(c) performing an in vitro transcription reaction using the DNA fragment product as a template to obtain a first double-stranded RNA product including an RNA polymerase promoter sequence at both ends; and
(d) treating the obtained first double-stranded RNA product including the RNA polymerase promoter sequence with a DNA-degrading enzyme and a single-strand-specific RNase to obtain a second double-stranded RNA,
wherein in (b), the PCR is performed by a large-scale PCR system,
wherein the large-scale PCR system comprises: a reaction vessel made of a heat-transferable material containing a PCR reaction mixture; and a first heat transfer vessel, a second heat transfer vessel, and a third heat transfer vessel, each capable of accommodating the reaction vessel and containing liquids set to temperatures for denaturation, annealing, and extension, respectively and each of the heat transfer vessel is capable of accommodating two or more of the reaction vessels,
wherein the PCR comprises moving the reaction vessel to the first heat transfer vessel, the second heat transfer vessel, and the third heat transfer vessel to perform denaturation, annealing, and extension reactions, respectively,
and further comprising, after (a) and before (c), introducing the recombinant vector into a microbial cell, and culturing the cell to construct a cell bank (CB) containing the recombinant vector,
wherein a concentration of the first double-stranded RNA obtained from the in vitro transcription reaction is 6 mg/mL or more.

2-4. (canceled)

5. The method for large-scale production of double-stranded RNA of claim 1, wherein the reaction vessel is made of a material capable of transferring heat of the liquid in the heat transfer vessel to the PCR reaction mixture in the reaction vessel when in contact with the liquid in the heat transfer vessel.

6. (canceled)

7. The method for large-scale production of double-stranded RNA of claim 1, wherein the reaction vessel contains 100 ml or more of the PCR reaction mixture.

8. The method for large-scale production of double-stranded RNA of claim 1, wherein the reaction vessel is made of a plastic material or a metal material.

9. The method for large-scale production of double-stranded RNA of claim 1, wherein the liquids in the heat transfer vessels are aqueous liquids.

10. The method for large-scale production of double-stranded RNA of claim 1, wherein the liquids are water.

11. The method for large-scale production of double-stranded RNA of claim 1, wherein the heat transfer vessel is in the form of a tank, a bath, or a chamber.

12. The method for large-scale production of double-stranded RNA of claim 1, wherein the PCR comprises:
initially denaturing the PCR reaction mixture at about 95° C. to about 96° C. for 5 minutes;
repeating denaturation, annealing and extension reactions 35 times to 38 times under conditions of about 95° C. to about 96° C. for about 1 minute to about 1 minute 30 seconds, about 58° C. to about 62° C. for about 1 minute 10 seconds to about 1 minute 40 seconds, and about 70° C. to about 74° C. for about 1 minute to about 1 minute 20 seconds, respectively for a product of the initial denaturation; and
performing a final treatment under conditions of about 71° C. to about 76° C. for 5 minutes.

13. (canceled)

14. The method for large-scale production of double-stranded RNA of claim 1, wherein a length of the double-stranded RNA is about 300 bp to about 1,500 bp.

15. The method for large-scale production of double-stranded RNA of claim 1, wherein the RNA polymerase promoter is a T7 promoter.

16. The method for large-scale production of double-stranded RNA of claim 1, wherein a buffer used in the in vitro transcription reaction has a pH of about 7.5 to about 8.0.

17-18. (canceled)
